(19) United States
(12) Patent Application Publication
(10) Pub. No.: US 2009/0162277 A1
(43) Pub. Date: Jun. 25, 2009

(54) Lysophospholipids Solubilized Single-Walled Carbon Nanotubes

(75) Inventors: Pu-Chun Ke, Clemson, SC (US); Yonnie Wu, Seneca, SC (US); Apparao M. Rao, Anderson, SC (US)

Correspondence Address: J. BENNETT MULLINAX, LLC, P. O. BOX 26029, GREENVILLE, SC, 29616-1029, US

(73) Assignee: Clemson University, Clemson, SC (US)

(21) Appl. No.: 12/084,275

(22) PCT Filed: Nov. 3, 2006
(86) PCT No.: PCT/US06/43056

§ 371 Date: Apr. 29, 2008

Related U.S. Application Data
(60) Provisional application No. 60/733,244, filed on Nov. 3, 2005.

(51) Int. Cl. A61K 9/14; C12Q 1/02; A61K 51/02; A61K 49/00

(52) U.S. Cl. 424/1.29; 424/489; 435/29; 424/9.1; 977/742; 977/774; 977/842

(57) ABSTRACT

Lipophilic compounds extracted from cell growth mediums, particularly lysophospholipids are used to solubilize single-walled nanotubes. The naturally occurring lysophospholipids were found to readily bond to the exterior wall of the single-walled nanotubes to enhance the biocompatibility of the single-walled nanotubes in therapeutic and diagnostic conditions. The solubilization protocol is simple, highly efficient, and results in a population of coated single-walled nanotubes which are highly stable.

### RELATED APPLICATIONS

This application claims the benefit of U.S. Provisional Application Ser. No. 60/733,244 filed on Nov. 3, 2005, and which is incorporated herein by reference.

### FIELD OF THE INVENTION

This invention is directed towards a method for solubilizing single-walled carbon nanotubes (SWNTs) in an aqueous solution and the resulting solubilized SWNTs. The solubilized single-walled carbon nanotubes may be used for a number of biological applications including direct delivery of biologically active agents, in vivo imaging, biodetection, and cell penetration.

### BACKGROUND OF THE INVENTION

The use of SWNTs as nanoagents for therapeutics, diagnostics, imaging, and other medical and animal uses requires that the SWNTs have some degree of solubility. A major hurdle for creating carbon nanotubes in liquid phase is their tendency to bundle, attributable to hydrophobic interactions, van der Waals forces, and the π-stacking among individual tubes. Prior efforts at dispersing SWNTs have employed organic solvents and aqueous solutions. Such techniques have also involved the non-covalent attachment of proteins, polymers, surfactants, and nucleic acids with various degrees of effectiveness. SWNTs have also been covalently functionalized through the esterification or amidation of acid-oxidized nanotubes and the use of sidewall covalent attachment of functional groups.

While such applications are useful in certain conditions, there are concerns of cytotoxicity and side effects associated with the use of surfactants, organic solvents, and residues. Further, there often are uncertainties in determining reaction efficiency and additional concerns of undesirable alternations to the physical and chemical properties of the carbon nanotubes. Further, the prior art techniques of SWNTs modification can lead to undesirable physical and chemical properties of the SWNTs.

Accordingly, there remains room for improvement and variation within the art.

### SUMMARY OF THE INVENTION

It is one aspect of at least one of the present embodiments to provide for a process and resulting product of SWNTs having a coating of lysophospholipids or single-chain phospholipids which increase solubility for SWNTs.

It is yet another aspect of at least one of the present embodiments of the invention to provide for a population of SWNTs having an arrangement of lysophospholipid striations along the exterior of the SWNT tube access.

It is yet another aspect of at least one of the present embodiments of the invention to provide for a population of SWNTs which has been solubilized in an aqueous lysophospholipid solution.

It is still a further aspect of at least one of the present embodiments of the invention to provide for a lysophospholipid solubilized population of SWNTs having a functionalized molecule attached to one or more head groups of the lysophospholipids associated with the SWNTs.

It is a further aspect of at least one of the present embodiments to provide for a method of using lysophospholipid solubilized SWNTs as a biocompatible carrier for a functionalized molecule, such molecules including quantum dots, antioxidants, dyes, markers, monoclonal antibodies, and pharmacologically active molecules.

It is a further aspect of at least one of the present embodiments to provide for a population of lysophospholipid solubilized SWNTs in which at least one of a surface of the SWNTs or the attached lysophospholipids further carries a functional group such as an imaging agent, a therapeutic agent, a diagnostic agent, or dye molecule.

It is a further aspect of at least one of the present embodiments to provide for a population of SWNTs which have been solubilized lipophilic compounds extracted from cell growth.

It is a further aspect of at least one of the present embodiments to provide for a population of SWNTs which exhibit solubility in aqueous solutions and which possess long-term stability.

It is a further aspect of at least one of the present embodiments to provide for a SWNT construct comprising a plurality of single-walled nanotubes, an exterior surface of the single-wall nanotubes having a coating of a lysophospholipid. The lysophospholipids useful in providing the construct may be selected from the group consisting of lysoglycerolphosphatidic acid, lysoglycerolphosphatidylcholine, lysoglycerolphosphatidylglycerol, lysoglycerolphosphatidylglycine, lysoglycerolphosphatidylethanolamine and combinations thereof.

It is a further aspect of at least one of the present embodiments to provide for a single-walled carbon nanotube in which an exterior surface of the nanotube has a coating of a lysophospholipid wherein the lysophospholipid contains a reactive head group having a ligand bonded thereto, the ligand selected from the group consisting of antibodies, proteins, quantum dots, radionuclides, hormones, co-factors, bioactive agents and combinations thereof.

It is a further aspect of at least one embodiment of the present invention to provide for a method of preparing solubilized single-walled carbon nanotubes comprising: providing single-walled carbon nanotubes; placing the single-walled carbon nanotubes into a solution of one type of lysophospholipids; and, sonicating the single-walled nanotubes and the solution of lysophospholipids, thereby providing a supply of lysophospholipid solubilized single-walled carbon nanotubes.

These and other features, aspects, and advantages of the present invention will become better understood with reference to the following description and appended claims.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1 sets forth the chemical structure of lysophospholipids LPC 18:0 LPA 16:0, and LPG 16:0 and a surfactant SDS.

FIGS. 2A through 2E set forth comparative solubility and functional data on phospholipid solubilized SWNTs.

FIGS. 3A through 3D are transmission electron microscope images of SWNT-LPC (A and C) and SWNT-LPG (B) complexes along with drawings of the lipid spiral wrapping around the tube access of a SWNT.

FIGS. 4A through 4H set forth confocal images of fixed macrophages incubated with SWNT-LPC and examined for apoptosis by APO-BrdU TUNEL assay.

FIGS. 5A and 5B are graphs setting forth mass spectral characterization of the phospholipids in cell growth medium RPMI supplemented with 15% FBS and in NB.

FIGS. 6A through 6G set forth additional confocal images of fixed macrophages incubated with SWNT-LPC examined for apoptosis by APO-BrdU TUNEL assay.

FIGS. 7A through 7F are photomicrographs demonstrating the uptake of rhodamine-lysophosphoethanolamine SWNTs.

FIGS. 8A through 8C set forth binding models and electron micrographs indicating the orientation and arrangement of the lysphospholipids on the surface of the SWNTs.

### DESCRIPTION OF THE PREFERRED EMBODIMENT

Nano scale materials have become important tools in medicine and animal science for imaging, diagnostics, and therapeutic agents. One form of a nano scale material includes SWNTs which have a number of desirable attributes. Often, when SWNTs are used as nano agents, the SWNTs have been in direct contact with a biological environment which is often undesirable. The present invention uses naturally occurring lysophospholipids to encapsulate and thereby solubilize SWNTs. The enhanced solubilization confers useful physical and chemical properties, thereby expanding the utility of the SWNTs as a biocompatible material.

For biological applications such as imaging, diagnostics, and therapeutics, the SWNTs can be further associated with one or more targeting ligands. Such ligands may be selected to be specifically bindable or associated with a pre-selected biological target. The function of the ligands is to cause the SWNTs to associate with or adhere to a specific biological structure or tissue. In this manner, other functions associated with the SWNT may be carried out.

By way of example, target ligands useful with the lysophospholipid solubilized SWNTs include antibodies, lectins, other proteins, nanoagents such as quantum dots, phosphorescent or fluorescent markers, radiodensity markers, and radionuclides. Such ligands are non-limiting examples of agents which can be associated with the lysophospholipid solubilized SWNTs.

As is known in the art, SWNTs may have ligands and other molecules or materials bonded either directly to an external surface of the SWNT or through the use of an appropriate bridge molecule such as a portion of the lysophospholipid described herein. Further, it is known in the art that the interior lumen of a SWNT may be filled with a biologically active material and used as a delivery or transport system for a targeted population of cells. The ability to use phospholipids and/or the external surface of a SWNT for binding ligands and other molecules is well established in the art as set forth in the following publications: U.S. Pat. No. 5,840,674 A—Covalent Microparticle-Drug Conjugates For Biological Targeting; EP 650371 A1—Covalent Polar Lipid-Peptide Conjugates For Biological Targeting; US 2004/0087482 A1—Micro-article-Drug Conjugates For Biological Targeting; and US 2006/0008461 A1—Microparticle-Drug Conjugates For Biological Targeting, these references being incorporated herein by reference in their entirety for all purposes.

Single walled carbon nanotubes (SWNTs) have found tremendous applications in materials science, electronics, nanophotonics, chemical and biosensing, and very recently, in gene and drug delivery. The bottleneck for many of these applications is the inherent insolubility of SWNTs due to their mutual interactions. Schemes to overcome this problem include binding of organic molecules to SWNTs and wrapping of SWNTs using surfactants and synthetic and biopolymers. However, many of these approaches are bio-incompatible and/or inefficient. As set forth herein, lysophospholipids, or single-chained phospholipids offer unprecedented solubility for SWNTs. Surprisingly double-chained phospholipids were found ineffective in rendering SWNTs soluble. Using transmission electron microscopy (TEM) the present invention demonstrates that lysophospholipids wrap SWNTs as striations whose size and regularity are affected by the polarity of the lysophospholipids. These findings shed light on the debate over the binding mechanism of amphiphilic polymers and cylindrical nanostructures and has implications on the design of novel supramolecular complexes and nanodevices.

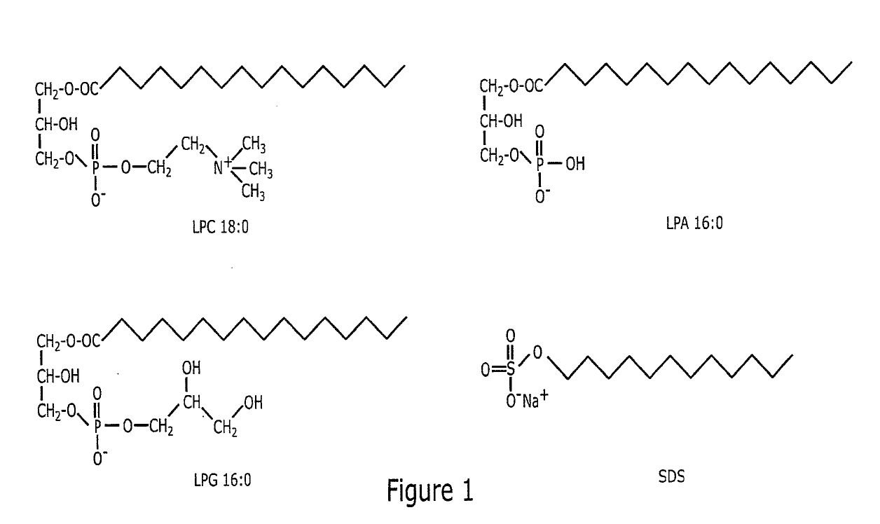

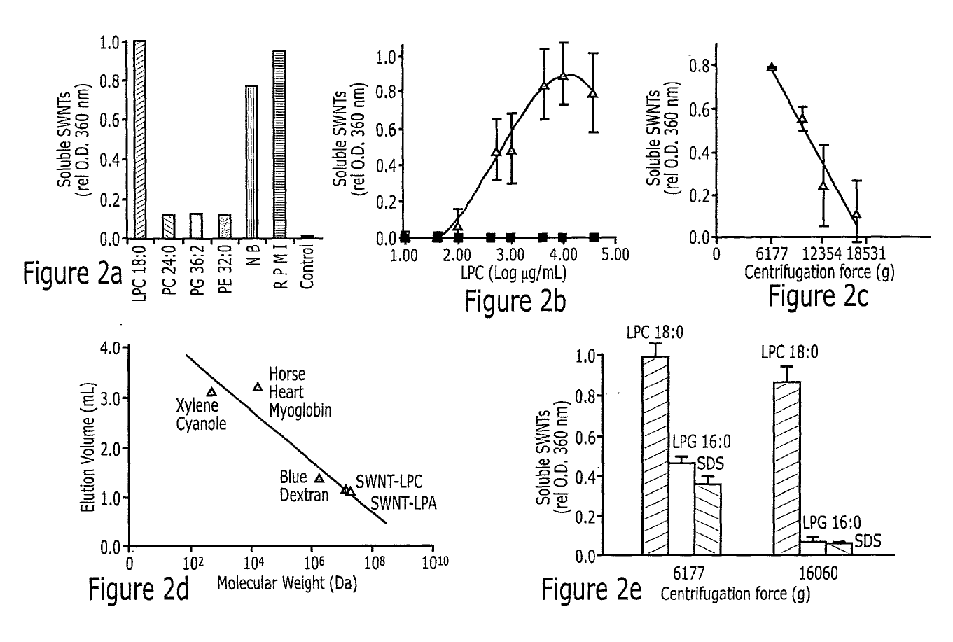

Pristine SWNTs were synthesized using arc-deposition method. The average diameter of the SWNTs was approximately 1.4 nm measured by Raman spectroscopy and the average molecular weight of the SWNTs was 1×10⁶ Dalton (Da) estimated from TEM. To measure the solubility provided by pure phospholipids, SWNTs were dispersed in phosphate buffered saline (PBS, pH=7.4) containing phospholipids of varying amounts. After sonication for 1 hr at room temperature, SWNTs were found completely solubilized by lysophosphatidylcholine, LPC 18:0 (FIG. 1, nomenclature see Supplementary Table 1), and by cell growth mediums (FIG. 2a). The weight ratio of solubilized SWNTs to LPC was approximately 1:10 corresponding to a molar ratio of 1:20,000 at saturation (FIG. 2b), indicating the high binding capacity of SWNTs. Comparable solubility of SWNTs was also obtained with lysophosphatidic acid, LPA 16:0 (FIG. 1), and lysophosphatidylglycerol, LPG 18:0 (FIG. 1), based on the same treatments.

Approximately half of the soluble SWNTs were sedimented at 16,060 g RCF at tip based on measured optical densities (FIG. 2c), indicating the heterogeneous size distribution of the SWNTs. From the elution volumes measured with chromatography, we estimated that the SWNT-LPC and the SWNT-LPA complexes had an average molecular weight of 14×10⁶ Da and 18×10⁶ Da, respectively (FIG. 2d). Since each SWNT carries approximately 20,000 LPC molecules and the molecular weight of each LPC is 523.7 Da, the “molecular weight” of each SWNT-LPC complex is estimated at 11.5×10⁶ Da which agrees with our measured value of 14×10⁶ Da. The discrepancy is mainly caused by the heterogeneous size distribution of the SWNTs and the occurrence of small SWNT bundles.

A comparison of SWNT solubility is given in FIG. 2e for LPC, LPG, and surfactant sodium dodecyl sulfate or SDS (FIG. 1), a routine solvent for SWNTs. At 6,177 g RCF at tip, on per molecule basis, LPC is approximately 2.5 times more effective than SDS in dispersing SWNTs in PBS. At 16,060 g RCF at tip, LPC is approximately one order of magnitude more effective than SDS in dispersing SWNTs possibly due to the fact that the resulting micelles differ in size. This difference might be because LPC has a bulkier head group for interfacing with water and a longer acyl chain for binding with SWNTs. The solubility of SWNTs with LPG is slightly better than SDS. In addition, solubilization of SWNTs with lysophospholipids was more effective than with nucleic acids, and far more effective than with proteins. The aqueous SWNT-lysophospholipid solutions were exceptionally stable for months at room temperature, a feature useful for their applications in biology and medicine.

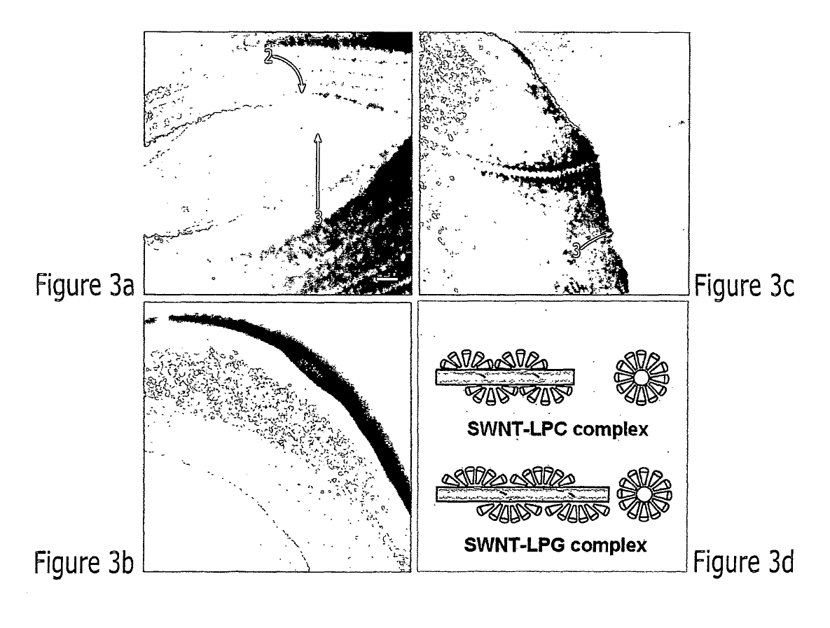

To probe the mechanism of SWNT-lysophospholipid binding, zwitterionic LPC and net negatively charged LPG at physiological pH were bound to SWNTs and imaged with TEM (FIGS. 3a-c). The Figures set forth the formation of areas of tightly packed lysophospholipids in the dark/grey areas, our termed “lipid phase”, in FIGS. 3a-c. The light/blank areas in FIGS. 3a-c correspond to lysophospholipid free regions or our termed “vacuum phase”. In the lipid phase SWNTs are wrapped by striations of 5 nm for LPC and 5-7 nm for LPG. However, in the vacuum phase (FIGS. 3a, c) SWNTs are practically naked indicating that the binding of lysophospholipids to SWNTs is controlled by the local lysophospholipid environment rather than by specific interactions between lysophospholipids and SWNTs.

Neither LPC nor LPG binds to SWNTs in the vacuum phase, while both coat SWNTs in the lipid phase. LPC on an SWNT or an SWNT bundle displays such a consistent organized pattern along the tube(s) that striations remain approximately the same size (FIGS. 3a, c). By contrast, the binding of LPG to SWNTs in the lipid phase does not follow the same pattern (FIG. 3b). The size and orientation of the striations change along the axis of SWNTs. These differences could be related to the different lysophospholipid organizations shown in their respective backgrounds. It can be seen (FIGS. 3a, c) that the lipid phase of LPC is composed of many large objects of ˜5 nm which probably are micelles, while the lipid phase of LPG is homogeneous, most probably composed of individual lysophospholipids. Another major difference in the binding of LPC vs. LPG in the lipid phase is the shape of the striations. The crests of LPG striations are about 0.2 nm above the surface of SWNT(s), while the clefts almost touch the surface of SWNT(s) for LPC.

To confirm the general observations, double-chained phospholipids were tested for their SWNT solubility. The phospholipids used are dimyristoyl phosphatidyl choline (PC 24:0) which is zwitterionic at physiological pH, and 1,2-dioleoylphosphatidylglycerol (PG 36:2) and 1,2-dipalmityolphosphatidylethanolamine (PE 32:0), both of which are negatively charged at physiological pH. None of the above phospholipids provided good solubility for SWNTs.

In addition to the packing consideration, the average number of LPC needed to coat an average SWNT was calculated assuming tight packing and the size of LPC head group of 0.6 nm. It was found that “half-cylinder” binding will result in 21,000:1 lipids/tube—a number that is in excellent agreement with the experimentally estimated ratio of 20,000:1.

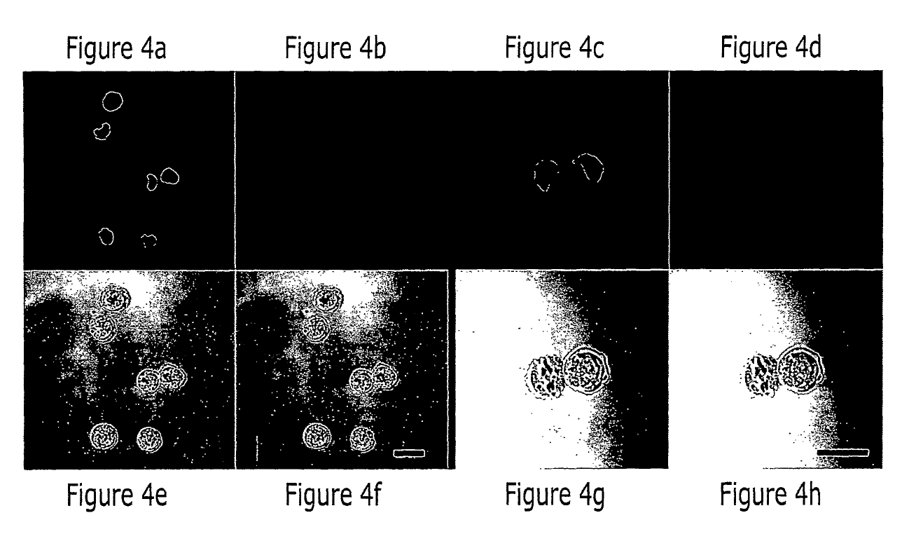

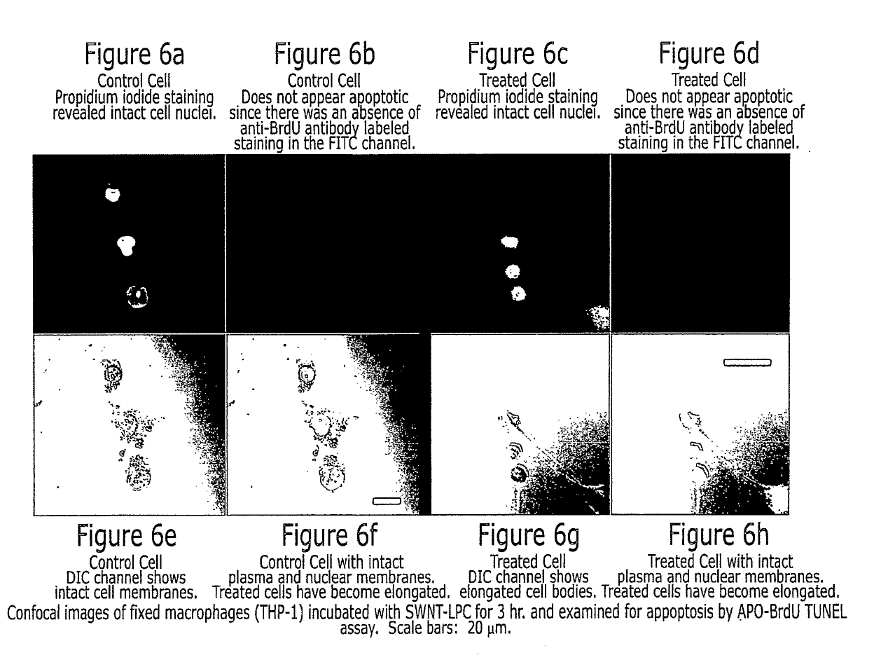

The bioassays further showed no loss of cell viability (FIG. 4 and FIG. 6) when both colon cancer cells (CACO-2) and macrophage (THP-1) cell lines were treated with 20 to 40 ppm of lysophospholipid-free and micelle-free SWNT-LPC. CACO-2 cell nuclei were unaffected by treatment of 20 ppm SWNT-LPC (FIG. 4c) which was also the case for the macrophage THP-1 cell line treated with 40 ppm SWNT-LPC (FIG. 6). Cell plasma membranes remained intact in CACO-2 cells (FIG. 4e) and in THP-1 cells (FIG. 6). The onset of apoptosis by Apo-BrdU TUNEL assay was not detected in either cell line (FIG. 4d and FIG. 6). Motile THP-1 cells treated at 40 ppm SWNT-LPC exhibited elongated cell bodies (FIG. 6) which are hypothesized to be due to SWNT-LPC disruption of plasma membranes or cytoskeleton.

SWNTs, otherwise a collection of hydrophobic synthetic nanoparticles, have been solubilized in aqueous lysophospholipid solutions with extended stability. The biocompatibility of lysophospholipids is unsurpassed since they occur naturally in the cell membrane. The signalling capacity of lysophospholipids and the electronic property of SWNTs may be combined for disease detection. The strong absorbance of isolated SWNTs in near infrared can be utilized for noninvasive imaging and sensing. Furthermore, since the head groups of lysophospholipids can be functionalized with tags such as quantum dots, antioxidants, and monoclonal antibodies, our method opens the door for utilizing nanomaterials for in vivo imaging, gene and drug therapy, and novel nanomedicine.

### Methods

Solubility Assays. Glycerol phospholipids and lysoglycerophopholipids were purchased from Avanti Polar Lipids, Inc, AL. Cell growth mediums NB and RPMI fortified with 10% FBS were obtained from Difco and Gibco (Invitrogen), respectively. SWNTs of 1 mg were placed in a series of eppendorf tubes containing lysophospholipids LPC 18:0, PC 24:0, PG 36:2, and PE 32:0 of 10, 40, 100, 400 μg and 1, 4, 10, 40 mg in 1 mL PBS solution. The eppendorf tubes were placed in a water bath and sonicated for 1 hr at room temperature. The solution was centrifuged for 3 min at 6,177 g. Approximately 0.3 mL of the supernatant was transferred into a glass cuvette and read for optical density against a blank at 360 nm.
Size Exclusion Chromatography. Sepharose CL-4B beads (Sigma) having an exclusion limit molecular weight of 20×10⁶ Da were used for gel filtration chromatography in a 10 mm (diameter)×150 mm (length) open column. The flow rate was produced by gravity at approximately 0.5 mL/min. Samples in 1×PBS of 40 μL were loaded onto the column with 1 M acetic acid as the mobile phase. Molecular weight markers with visible colours were used for calibration. The dark colour of the lysophospholipid dispersed SWNTs was monitored visually by comparing the elution volume with those of standard compounds. SWNT-LPC complexes were centrifuged at 16,060 g and SWNT-LPA complexes centrifuged at 6,177 g for 3 min. Their supernatants were used for size exclusion chromatography.
TEM Experiment. Buffered solutions of SWNT-LPC and SWNT-LPG were sonicated for 1 min. The solutions were placed on holey carbon grids for 1 min and the excess drawn off with filter paper. The grid was negatively stained with a 2% uranyl acetate solution for 1 min. The images were recorded at magnification ranges from 400,000 to 600,000 times with the Hitach 7600 transmission electron microscope at 100 and 120 kV.
Bioassay. Free lysophospholipids and micelles in SWNT-lysophospholipid solution were removed by filtration through 100 kDa Microcon (Amicon, Inc) centrifugation tubes and washed 4 times. The resulting lysophospholipid-free and micelle-free SWNT-LPC complexes were tested by in vivo bioassay using colon cancer (CACO-2) and macrophage (THP-1) cell lines. Each cell line was incubated in its own 8-well chamber slide (LabTek) for 48 hr at 37° C. in a CO₂ incubator. Prior to treatment, cell adhesion was checked by differential interference contrast (DIC) microscopy using a Zeiss 135 Axiovert inverted microscope. Treatments ranging from 5 to 40 ppm of lysophospholipid-free and micelle-free SWNT-LPC complexes were added to each adherent cell line and incubated for 3 hr at 37° C. in a CO₂ incubator. After incubation, control and treated cells were fixed with 4% paraformaldehyde for 30 min, washed in PBS and subjected to an APO-BrdU TUNEL assay (Invitrogen). This assay detects the onset of apoptosis by fluorescent detection of nuclear DNA fragments or DNA breaks. Cells were labeled with deoxythymidine analog 5-bromo-2′-deoxyuridine 5′ triphosphate (BrdUTP) followed by the addition of Alexa-Fluor 488 labeled anti-BrdU antibody. Propidium iodide was used to image the total DNA content of cells. The prepared cells were imaged using a Zeiss 510 LSM confocal fluorescence microscope.

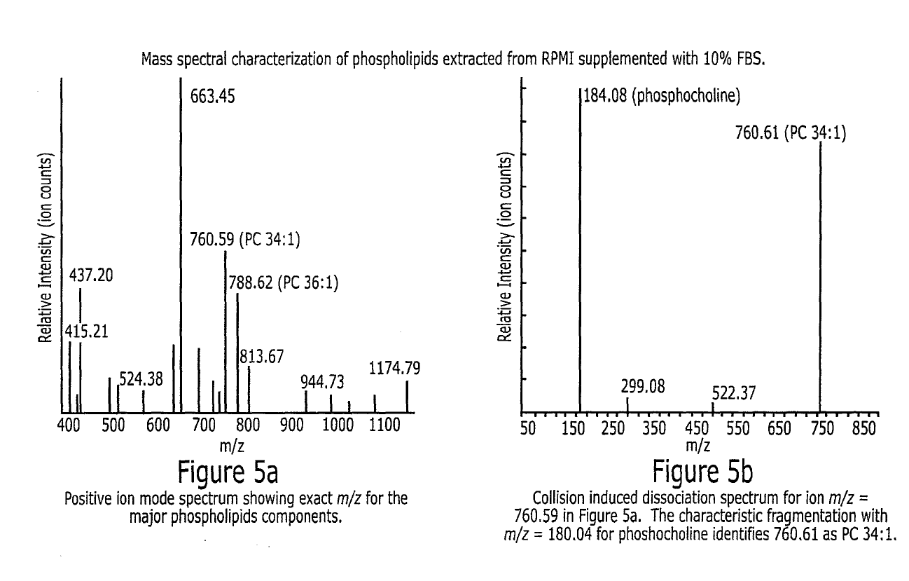

Mass Spectrometry. For mass spectroscopic characterization of cell growth mediums RPMI supplemented with 10% FBS and NB, both positive and negative ion mode acquisitions were performed for anionic and cationic lipids. The phospholipid species in cell growth mediums were identified by product ion scan where signature fragments corresponding to specific head groups, phosphoric acids, and acyl chains were revealed. A typical precursor ion spectrum of positive lipids extracted from RPMI fortified with 10% FBS is illustrated in FIG. 5a. The product ion spectrum corresponding to the peak of 760.59 in FIG. 5a is exemplified in FIG. 5b. The characteristic fragmentation of ion 760.61 corresponds to the phospholipid PC 34:1. Following the same procedures, we identified 10 major phospholipids (Table 1) in both the positive and negative ion mode for RPMI plus FBS and for NB. The chemical properties of the identified phospholipids vary in polarity, charge, and the number and the length of fatty acyl chains. Screening of common phospholipids was possible by matching their mass m over charge z with known phospholipid tables. The majority of the lysophospholipids, once bound to SWNTs, were found to be extractable only by multiple treatments with methanol:chloroform indicating strong interaction.

Both positive and negative ion mode acquisitions were performed using the quadrapole time-of-flight mass spectrometer (Q-T of Micro™) with capillary HPLC and electrospray ion source (Waters Corp., Milford, Miss.) using Masslynx software (V4.0, Waters Corp., Milford, Miss.). The sample was directly injected from the sample vial into the electrospray ion source with 10 min acquisition using a mobile phase of methanol:chloroform=50/50 (v/v) containing 0.1% formic acid or 0.1% NH₄OH at a flow rate of 1.5 μL/min. Mass scanning range was 400 to 1200 mass units per 1 sec with a 0.1 sec inter-scan delay in continuum mode. Glu-fibrinopeptide was used for calibration in MS and MS/MS mode and infused through the nanoLockspray for single point external mass calibration in both positive and negative ion mode at 784.8426 and 782.8426 Da, respectively. Raw spectra were processed using MassLynx. A full scale intensity threshold of 0.1% was set and the peak lists containing m/z and intensity are set for below in Table 1.

TABLE 1

| RPMI +10% FBS | | | | NB | | | |
|---|---|---|---|---|---|---|---|
| Positive Lipid (M + 1) | | Negative Lipid (M − 1) | | Positive Lipid (M + 1) | | Negative Lipid (M − 1) | |
| Name | Rel. Quantity | Name | Rel. Quantity | Name | Rel. Quantity | Name | Rel. Quantity |
| PC 34:1 | 102.2 | LPG-H 16:0 | 40.8 | SPM 34:1 | 17.0 | LPA 16:0 | 21.9 |
| PC 36:1 | 69.6 | LPA 16:0 | 36.4 | PCp 38:4 a/o PCe 38:5 | 16.5 | LPA 18:0 | 13.5 |
| SPM 34:1 | 40.2 | PEp 38:6 | 34.5 | PCe 36:0 | 16.0 | LPE 14:0 | 6.3 |
| PC 36:2 | 29.6 | LPI 16:0 | 21.8 | SPM 34:2 | 15.8 | LPE 16:1 | 6.6 |
| PCe 28:0 | 26.4 | LPA 18:0p | 21.8 | PCe 28:0 | 13.9 | LPE 18:1 | 4.9 |
| SPM 42:2 | 19.8 | PE 38:3 | 17.1 | SPM 35:1 dihydroxy | 6.9 | LPE 18:0 | 4.7 |
| PC 32:0 | 19.5 | PEe 36:5 | 12.9 | PCp 40:1 a/o PCe 40:2 | 6.2 | LPE 20:3 | 8.6 |
| PC 38:4 | 17.9 | LPS 18:0 | 11.5 | SPM 42:0 | 5.1 | LPS 18:1 | 8.0 |
| PC 38:3 | 17.5 | PS 38:6 | 10.7 | SPM 42:0 dihydroxy | 4.8 | LPS 18:0 | 7.2 |
| LPC 18:0 | 16.6 | PE 36:4 | 10.0 | SPM 40:2 | 3.9 | LPS 18:0 | 5.1 |

As seen in Table 1, major positive and negative phospholipids and lysophospholipids in cell growth mediums RPMI supplemented with 10% FBS and NB are identified. PA denotes phosphatidic acid, LPA lysophosphatidic acid, PC phosphatidylcholine, PCp plasmanyl phosphatidylcholine, PCe plasmenyl phosphatidylcholine, PE phosphatidylethanolamine, PS phosphatidylserine, PG phosphatidylglycerol, and PI phosphatidylinositol. The numbers “34” and “1” in PC 34:1 denote the total number of carbon atoms and the total number of double bonds contained in the sum of the fatty acyl chains respectively.

The lysophospholipid solubilized single-walled carbon nanotubes described herein provide a useful vehicle for introducing biologically active ligands into cells, tissues, and organs. The resulting coated SWNTs may be used with a variety of ligands including but not limited to proteins, antibodies, glycoproteins and lectins, peptides, polypeptides, saccharides, vitamins, steroids, steroid analogs, hormones, co-factors, bioactive agents, and genetic material including nucleosides, nucleotides, and polynucleotides. The ligands can be used to specifically target receptors on or near selected biological targets. As used herein, the term “receptor” refers to a molecular structure within the cell or on the surface of the cell which is generally characterized by the selective binding of a specific substance. Exemplary receptors may include cell-surface receptors for peptide hormones, neurotransmitters, antigens, complement fragments, immunoglobulins, and cytoplasmic receptors. As is known in the art, receptors may be membrane bound, cytosolic, or nuclear, monomeric, or multimonomeric. For some embodiments, the receptor may be a target protein on or near a selected biological cell, tissue, organ, or tumor.

One therapeutic use for the lysophospholipid SWNTs makes use of the solubilized SWNT which has an appropriate ligand applied to either an interior lumen of the SWNT, an exterior portion of the SWNT wall, or attached to the lysophospholipid. The resulting construct allows for a treatment protocol of a localized vascular tumor as well as a more systemic disease such as leukemia. The resulting construct may be localized by immunochemical bonding using appropriate ligands. Alternatively, the construct may utilize the “leaky endothelium” properties of tumor cells in which the constructs may more readily enter into the interior of an abnormal cell. The resulting construct, including appropriate ligand, can be used for visualization as well as treatment opportunities by delivering an appropriate dose of radiation, anti-tumor drug, or other conventional radioimmunotherapeutic agents.

It is believed that the constructs envisioned herein have sufficient shape and physical properties such that when introduced into a patient's body, the construct will not cross the blood-brain barrier, thereby reducing the risk that a therapeutic agent will be delivered to an unintended location within the body.

Additional therapeutic uses may take advantage of the ability of a single-walled carbon nanotube to be an agent for localized heating within a cell. Upon exposure to a proper stimuli, such as an infrared laser beam, target cell having a sufficient concentration of the solubilized SWNTs can be heated to a temperature which results in cell death. The ability to target individual cells allows for an effective treatment protocol which minimizes damage to surrounding populations of non-target cells.

Additional details regarding the protocol and methodology directed to the formation, characterization, and properties of the solubilized SWNT's may be seen in reference to the publication titled, Detection of Phospholipid-Carbon Nanotube Translocation Using Fluorescence Energy Transfer, Applied Physics Letter 89, 143-148 (2006); and Coating Single-Walled Carbon Nanotubes With Phospholipids, The Journal of Physical Chemistry B, 2006, 110, 2475-2478, both of which are incorporated herein by reference.

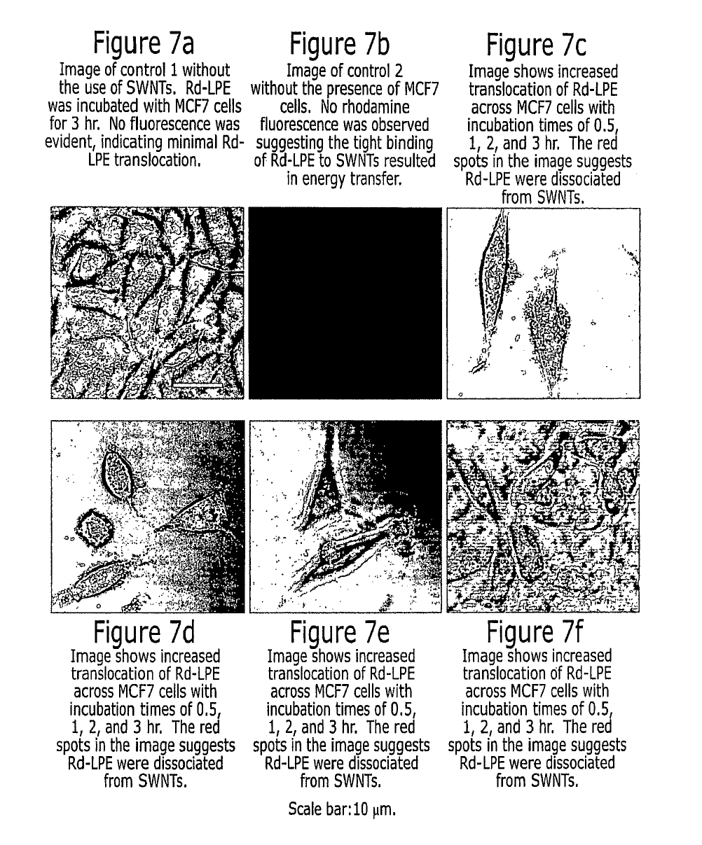

The SWNT lipid assemblies described above may be used to form an “optical switch” using rhodamine-lysophosphoethanolamine (Rd-LPE) and SWNTs. In accordance with this invention, it has been found that the Rd-LPE molecule will donate its absorbed energy to its acceptor of an associated SWNT. The construct of a Rd-LPE molecule and a coupled SWNT functions as a fluorescence resonance energy transfer (FRET) pair. As seen in reference to FIGS. 7A-F, when coupled together, the Rd-LPE and SWNT combination demonstrated a noticeable quenching of the rhodamine fluorescence. FIG. 7A sets forth a control image without SWNTs. Rd-LPE was incubated with MCF7 cells for three hours. No fluorescence was evident, indicating minimal Rd-LPE translocation as seen in image 7B. Such results indicate that the lack of rhodamine fluorescence suggests the tight binding of Rd-LPE to SWNTs resulted in energy transfer. Images 7C through 7F show increased translocation of Rd-LPE across the MCF7 cells with respective incubation times of 0.5, 1.0, 2.0, and 3.0 hours. The red spots in the images suggest Rd-LPE was disassociated from the SWNTs following translocation across the cell membranes. While control cells indicated no intracellular rhodamine fluorescence, the incubated cells demonstrated high fluorescence levels in the cell cytophages when viewed at a number of different focal depths suggesting a high translocation efficiency of the Rd-LPE SWNT complexes across the cell membrane. Further, the fluorescence is indicative that a physical separation between the SWNT and the Rd-LPE has occurred since the quenching effect was not observed.

This data indicates that solubilized SWNTs are able to cross cell membranes while carrying a functional molecule such as rhodamine. The Rd-LPE-SWNT assembly also provides a visible marker indicating that the assembly may be transported across the cell membrane and in materials released from the lipid portion of the molecule. Accordingly, it provides a “optical switch” or visual indicator of transport of the SWNT indicating the lipid head portions did cross the cell membrane. As is readily appreciated by one having ordinary skill in the art, the head portions of the lipids provide a number of active binding sites to which materials such as rhodamine, antibodies, nucleic acids, genes, prodrugs, drugs, and contrast agents (for enhancing magnetic resonance imaging) can be transported across cell membranes. The lipids provide a number of binding sites suitable for interactions including conjugations, polar bonding, covalent bonding, and/or the use of linking bridge molecules so as to bring about association of a functional molecule with the lipid portion of the solubilized SWNT.

The presence of strong rhodamine fluorescence in intracellular spaces demonstrates a simple and efficient release of an active molecule from a SWNT carrier. The use of the lipid solubilization moleules to transport active molecules represents a tremendous advance in introducing and thereafter releasing biological agents within living cells. Conventional covalent bonding methods using nanotubes and other nanocarbon structures are not able to achieve the effective release mechanism demonstrated here.

### Example 2

Fullerene C₇₀ was coated with gallic acid which emits green autofluorescence. The resulting C₇₀ complexes can be used for visualizing localized nanomaterials in cells and living organisms. Following incubation of the gallic acid treated Fullerene C₇₀, the subsequent fluorescence of a daphnid appeared to be localized in the cell membranes. While not wanting to be limited by theory, it is Applicant's belief that the use of solubilized Fullerenes will provide similar specificity for directing lysophospholipid-LPC coated materials to a cell membrane which offers advantages for certain drugs, therapeutic treatments, and investigations. Fullerene C₆₀ has been solubilized by lysophospholipids LPC in aqueous solutions. Further, a CHO cell line is incubated with the coated Fullerene C₆₀ at a concentration of 0.6 mg/ml and an incubation time of 4 hours. The resulting Fullerenes emit fluorescence when excited with a laser. The location of the solubilized Fullerenes can be detected within both the membranes and cytoplasm of the CHO cells.

### Example 3

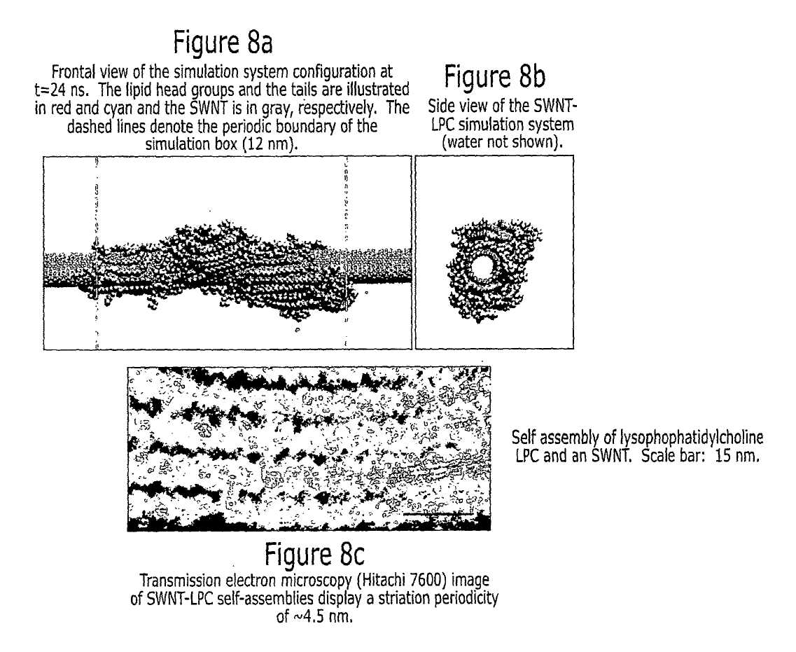

In accordance with this invention, it has been found that the binding pattern of lipids onto a SWNT achieves an organized structure much different from many models theorized in the literature such as the model set forth in FIG. 3D. As seen in reference to FIGS. 8A through 8C, FIGS. 8A and 8B show respective front and side views of lipids associated with a SWNT using the techniques described herein. As seen in FIG. 8A, the lipid bump I is believed formed from the gradual adsorption of lipids from a bulk supply while the lipid bump II is formed from the adsorption of a lipid cluster. The lipid head groups and tails are illustrated in respective red and cyan and the SWNT in gray. FIG. 8C is a TEM image of a SWNT-LPC assembly which displays a striation periodicity of 4.5 nm. The reference scale bar is 15 nm. The striation periodicity conforms to the predicted structures in the simulation seen in FIGS. 8A and 8B. As seen in FIGS. 8A and 8B, it is believed that the lipid tails are aligned approximately with the tube axis which therefore maximizes their mutual hydrophobic interaction. This arrangement also allows the lipids on the tube to disassociate and allows lipids in solution to bind to the tube at later stages.

### Example 4

Another useful feature of the lysophospholipid coated SWNTs is that the nanotubes, once coated, avoid the tendency to form clumps. Uncoated nanotubes or nanotubes that have significant exposed surfaces will tend to bind with other nanotubes to form large, random structures. Within living systems, the clumped or aggregated, uncoated nanotubes are believed to interfere with normal biotic processes within a cell. For instance, there are numerous published reports directed to the toxicity of nanotubes. It is believed that many of the toxicity studies are not related to inherent toxicity of the nanotubes per se, but rather reflect deleterious effects when nanotubes are aggregated into large clumps. In such aggregates, clumps can interfere with normal cellular processes including interfering with cytoskeleton assisted functions such as mytosis or myosis. Large aggregations of nanotubes can also interfere with intracellular transport of materials. In addition, at a tissue and/or an organism level, aggregates of nanotubes can form aggregates that interfere with larger scale functions such as feeding, the absorption of foods, and for single-celled organisms, uncoated nanotubes can physically bind to the organism to such an extent that normal motility is prevented resulting in the death of the organism. In accordance with this present invention, it has been found that the phospholipid coated SWNTs described herein are resistant to clumping. Accordingly, it is believed that not only are enhanced levels of nanotube accumulation in cells possible without deleterious effects, but that the ability to coat nanotubes affords a unique approach in testing the toxicity of nanotubes and other carbon nanostructures.

While the above examples make use of solubilized SWNTs, it is believed that a wide number of carbon nanostructures can be successfully solubilized so as to render the corresponding carbon substrate suitable for various biodelivery techniques and systems. While not separately reported, it has been observed that when the lipid solubilization techniques described herein are applied to carbon sheets, solubilization is noted by virtue of the characteristic color change of the solution. These solubilized carbon sheets are believed to represent single solubilized sheets or layers of a small number of joined sheets which are soluble by the binding of the lysophospholipids. It is believed that the formation of solubilized layer sheet(s) offer a useful template for nanoelectronics as well as for attaching a wide variety of agents which can be incorporated into living cells for various therapeutic and diagnostic protocols.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention which is set forth in the following claims. In addition, it should be understood that aspects of the various embodiments may be interchanged both in whole or in part. Therefore, the spirit and scope of the appended claims should not be limited to the description of the preferred versions contained therein.

Set forth in the accompanying Appendix is a list of publications directed to various aspects of carbon nanotube properties and techniques for using and analyzing carbon nanotubes. Many of the references set forth protocols which are well known in the art. These references are hereby incorporated herein by reference for all purposes.

### APPENDIX

1. Kong, J. et al. Nanotube molecular wires as chemical sensors. Science 287, 622-625 (2000).
2. O'Connell, M. J. et al. Band gap fluorescence from individual single-walled carbon nanotubes. Science 297, 593-596 (2002).
3. Chen, R., Zhang, Y., Wang, D. & Dai, H. Noncovalent sidewall functionalization of single-walled carbon nanotubes for protein immobilization. J. Am. Chem. Soc. 123, 3838-3839 (2001).
4. Shim, M., Kam, N. W. S., Chen, R. J. & Dai, H. Functionalization of carbon nanotubes for biocompatibility and biomolecular recognition. Nano Lett. 2, 285-288 (2002).
5. Chen, R. J. et al. Noncovalent functionalization of carbon nanotubes for highly specific electronic biosensors. Proc. Natl. Acad. Sci. USA 100, 4984-4989 (2003).
6. Barone, P. W., Baik, S., Heller, D. A. & Strano, M. S. Near-infrared optical sensors based on single-walled carbon nanotubes. Nature Mater. 4, 86-92 (2005).
7. Hu, H., Ni, Y., Montana, V., Haddon, R. C. & Parpura, V. Chemically functionalized carbon nanotubes as substrates for neuronal growth. Nano Lett. 4, 507-511 (2004).
8. Kam, N. W. S., Jessop, T. C., Wender, P. A. & Dai, H. Nanotube molecular transporters: internalization of carbon nanotube-protein conjugates into mammalian cells. J. Am. Chem. Soc. 126, 6850-6851 (2004).
9. Bianco, A., Kostarelos, K., Partidos, C. D. & Prato, M. Biomedical applications of functionalised carbon nanotubes. Chem. Commun. 5, 571-577 (2005).
10. Pantarotto, D., et al. Immunization with peptide-functionalized carbon nanotubes enhances virus-specific neutralizing antibody responses. Chem. Biol. 10, 961-966 (2003).
11. Lu, Q. et al. Nano Lett. RNA polymer translocation with single-walled carbon nanotubes. 4, 2473-2477 (2004).
12. Kam, N. W. S., O'Connell, M., Wisdom, J. A. & Dai, H. Carbon nanotubes as multifunctional biological transporters and near-infrared agents for selective cancer cell destruction. Proc. Natl. Acad. Sci. USA 102, 11600-11605 (2005).
13. O'Connell, M. J. et al. Reversible water-solubilization of single-walled carbon nanotubes by polymer wrapping. Chem. Phys. Lett. 342, 265-271 (2001).
14. Pompeo, F. & Resasco, D. E. Water solubilization of single-walled carbon nanotubes by functionalization with glucosamine. Nano Lett. 2, 369-373 (2002).
15. Sun, Y., Wilson, S. R. & Schuster, D. I. High dissolution and strong light emission of carbon nanotubes in aromatic amine solvents. J. Am. Chem. Soc. 123, 5348-5349 (2001).
16. Zheng, M. et al. DNA-assisted dispersion and separation of carbon nanotubes. Nature Mater. 2, 338-342 (2003).
17. Yureli, K., Mitchell, C. A. & Krishnamoorti, R. Small-angle neutron scattering from surfactant-assisted aqueous dispersions of carbon nanotubes. J. Am. Chem. Soc. 126, 9902-9903 (2004).
18. Matarredona, O. et al. Dispersion of single-walled carbon nanotubes in aqueous solutions of the anionic surfactant NaDDBS. J. Phys. Chem. B 107, 13357-13367 (2003).
19. Richard, C., Balavoine, F., Schultz, P., Ebbesen, T. W. & Mioskowski, C. Supramolecular self-assembly of lipid derivatives on carbon nanotubes. Science 300, 775-778 (2003).
20. Strano, M. S. et al. Understanding the nature of the DNA-assisted separation of single-walled carbon nanotubes using fluorescence and Raman spectroscopy. Nano Lett. 4, 543-550 (2004).
21. Lichtenberg, D., Goni, F. M. & Heerklotz, H. Detergent-resistant membranes should not be identified with membrane rafts. Trends Biochem. Sci. 30, 430-436 (2005).
22. Milne, S. B., Forrester, J. S., Ivanova, P. T., Armstrong, M. D. & Brown, H. A. Multiplexed lipid arrays of anti-immunoglobulin M-induced changes in the glycerophospholipid composition of WEHI-231 cells. AfCS Research Reports 1, 1-11 (2003).

1. A composition comprising a plurality of single-walled nanotubes, an exterior surface of said single-walled nanotubes having a coating of a lysophospholipid.

2. The composition according to claim 1 wherein the lysophospholipid is selected from the group consisting of lysoglycerolphosphatidic acid, lysoglycerolphosphatidylcholine, lysoglycerolphosphatidylglycerol, lysoglycerolphosphatidylglycine, lysoglycerolphosphatidylethanolamine and combinations thereof.

3. The composition according to claim 1 wherein said lysophospholipid contains a reactive head group having a ligand bonded thereto, said ligand selected from the group consisting of antibodies, proteins, dyes, quantum dots, radionuclides, hormones, co-factors, bioactive agents and combinations thereof.

4. A method of preparing solubilized carbon nanostructures comprising:
providing a supply of carbon nanostructures;
placing the carbon nanostructures into a solution of lysophospholipids; and,
sonicating said carbon nanostructures and said solution of lysophospholipids, thereby providing a supply of lysophospholipid solubilized carbon nanostructures.

5. A process of increasing the solubilization of a carbon nanostructure comprising:
providing a supply of carbon nanostructures;
placing the carbon nanostructures into a solution of lysophospholipids; and,
sonicating said carbon nanostructures in said solution of lysophospholipids, thereby providing a supply of lysophospholipids solubilized carbon nanostructures;
wherein solubilized carbon nanostructures are able to be translocated across a membrane of a cell.

6. A composition comprising a single-walled nanotube, an exterior surface of said single-walled nanotube having a coating of a lysophospholipid wherein the lysophospholipid assembly has a plurality of striations having a periodicity of about 4.5 nm of the encapsulating lipids covering said single-walled nanotube.

7. A method of monitoring the update of a lysophospholipid coated single-walled nanotube comprising:
applying a rhodamine molecule to the lipid portion of a lysophospholipid coated single-walled nanotube, said single-walled nanotube substantially quenching a fluorescence of said rodamine molecule when bonded to said lysophospholipid coated single-walled nanotube;
introducing said rhodamine labelled lysophospholipid coated single-walled nanotube into a living cell, said rhodamine labelled lysophospholipid coated single-walled nanotube passing through a membrane of said living cell into a cytoplasm of a cell where the rhodamine molecule is at least partially disengaged from said lysophospholipid coated single-walled nanotube;
monitoring the fluorescence of the rhodamine molecule within the cell cytoplasm, said rhodamine fluorescence being indicative of movement of the lysophospholipid coated single-walled nanotube into the cell.

8. An assay for determining the toxicity of a nanostructure comprising:
providing a supply of carbon nanostructures;
attaching thereto a coating of lysophospholipids, thereby providing a supply of lysophospholipids solubilized carbon nanostructures; and,
conducting toxicity studies using the coated nanostructures.

9. The process according to claim 8 wherein said toxicity study is an LD 50 study.
